(19) United States
(12) Patent Application Publication
(10) Pub. No.: US 2010/0167227 A1
(43) Pub. Date: Jul. 1, 2010

(54) TOOTH WHITENING APPARATUS AND HEAD GEAR FOR TOOTH WHITENING APPARATUS

(76) Inventor: Jong-Chan Jun, Gyeonggi-do (KR)

Correspondence Address: Jae Y. Park, Kile, Goekjian, Reed & McManus, PLLC, 1200 New Hampshire Ave. NW, Suite 570, Washington, DC, 20036, US

(21) Appl. No.: 12/676,576

(22) PCT Filed: Sep. 7, 2007
(86) PCT No.: PCT/KR07/04353

§ 371 Date: Mar. 4, 2010

(51) Int. Cl. A61N 5/00

(52) U.S. Cl. 433/29

(57) ABSTRACT

Provided is a tooth whitening apparatus that allows a patient receiving tooth whitening treatment to move freely. By allowing a patient to wear the tooth whitening apparatus like a headphone, the patient is provided with the convenience of being able to move both hands freely and move around during tooth whitening treatment.

### TECHNICAL FIELD

The present disclosure relates to a tooth whitening apparatus and head gear for a tooth whitening apparatus. More particularly, the present disclosure relates to a tooth whitening apparatus and head gear for a tooth whitening apparatus that enable a patient to wear the apparatus together with the head gear and move freely while receiving tooth whitening treatment.

### BACKGROUND ART

As interest in plastic surgery and beauty continues to grow, tooth color has become an issue for personal appearance. Teeth can become discolored from their original color to yellow or darker hues due to any number of reasons, including side effects of drugs, congenital factors, smoking, and food coloring. Whitening teeth is considered by most young women today as a beautifying means.

To whiten teeth, however, much time and money must be invested, pain must often be endured over a prolonged period of whitening treatment, and patients are often dissatisfied with the whitening results after having endured extended painful treatment.

In a tooth whitening treatment according to the related art, a patient must lie still for a long time in a treatment chair while receiving treatment, which inconveniences both the patient and the dentist.

### DISCLOSURE OF INVENTION

### Technical Problem

Embodiments provide a tooth whitening apparatus that can be worn by a patient like headphones, to alleviate the discomfort of tooth whitening treatment according to the related art.

Embodiments also provide a tooth whitening apparatus that allows a patient to freely move his/her hands and head while receiving tooth whitening treatment wearing headphone-style head gear, thus permitting bodily movement and providing convenience.

Embodiments also provide a tooth whitening apparatus capable of maximizing efficiency of tooth whitening treatments by providing both patients and doctors with convenience during treatment.

### Technical Solution

In one embodiment, a head gear for a tooth whitening apparatus, the head gear includes: a side support contacting a side of a head; and a tooth whitening apparatus support connected to the side support and on which the tooth whitening apparatus is installed.

In another embodiment, a head gear for a tooth whitening apparatus, the head gear includes: a top support contacting and fixed on a top of a head; a rear support contacting and fixed at a rear of the head; a coupler connecting the top support to the rear support; a side support connected to the coupler and contacting a side of the head; and a tooth whitening apparatus support connected to the side support and on which the tooth whitening apparatus is installed.

In further another embodiment, a tooth whitening set includes: a side support contacting a side of a head; a tooth whitening apparatus support connected to the side support; and a tooth whitening apparatus installed on the tooth whitening apparatus support to radiate light on teeth.

### ADVANTAGEOUS EFFECTS

An advantage of the tooth whitening apparatus and head gear according to the present disclosure is that because a patient is able to wear the apparatus like a headphone, the patient is able to freely move both arms and the head region while receiving tooth whitening treatment. By thus enabling a degree of bodily movement, boredom can be reduced, and stiffness of the neck and shoulders (often following tooth whitening treatment) can be alleviated.

Another advantage of the tooth whitening apparatus and head gear according to the present disclosure is that because the apparatus and head gear provide comfort to both patient and doctor during tooth whitening treatment, the effects of the tooth whitening treatment can be maximized.

### BRIEF DESCRIPTION OF THE DRAWINGS

FIG. 1 illustrates a perspective view of head gear for a tooth whitening apparatus according to an embodiment of the present disclosure.

FIG. 2 illustrates a frontal view of the head gear in FIG. 1 viewed from a direction A.

FIG. 3 illustrates a rear view of the head gear in FIG. 1 viewed from a direction B.

FIG. 4 illustrates a top view of the head gear in FIG. 1 viewed from a direction C.

FIG. 5 illustrates a side view of the head gear in FIG. 1 viewed from a direction D.

FIG. 6 illustrates a perspective view of the head gear in FIG. 1 illustrating examples of its movement.

FIG. 7 illustrates a structural view of a side support according to an embodiment of the present disclosure.

FIG. 8 illustrates a cap that couples to the side support in FIG. 7.

FIG. 9 illustrates a side view of the cap in FIG. 8.

FIG. 10 illustrates a perspective view of a tooth whitening apparatus installed on a head gear, according to an embodiment of the present invention.

FIG. 11 illustrates a perspective view of the tooth whitening apparatus according to the present disclosure.

FIGS. 12 and 13 respectively illustrate a plan view and a sectional view of a light emitting portion forming a tooth whitening apparatus according to the present disclosure.

FIGS. 14 and 15 illustrate light emitting portions of tooth whitening apparatuses according to different embodiments of the present disclosure.

FIGS. 16 and 17 respectively illustrate a sectional view of a light emitting portion of a tooth whitening apparatus according to other embodiments of the present disclosure.

FIGS. 18 and 19 respectively illustrate a perspective view and a sectional view of a body of a light emitting portion forming a tooth whitening apparatus according to other embodiments of the present disclosure.

### BEST MODE FOR CARRYING OUT THE INVENTION

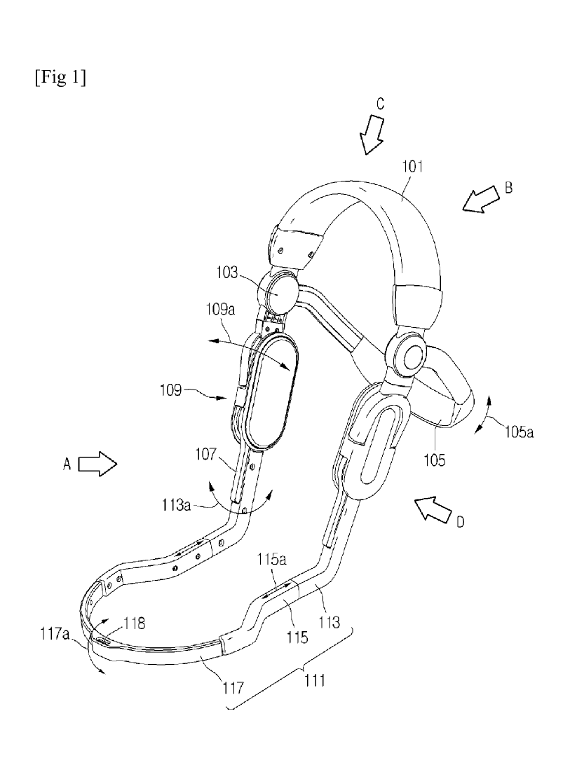

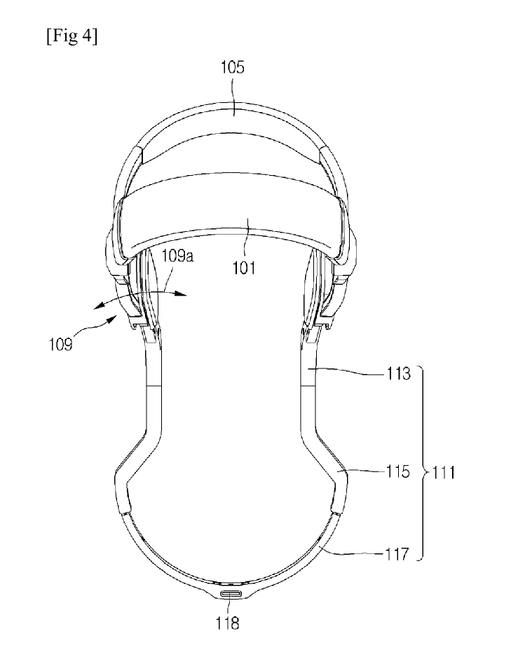

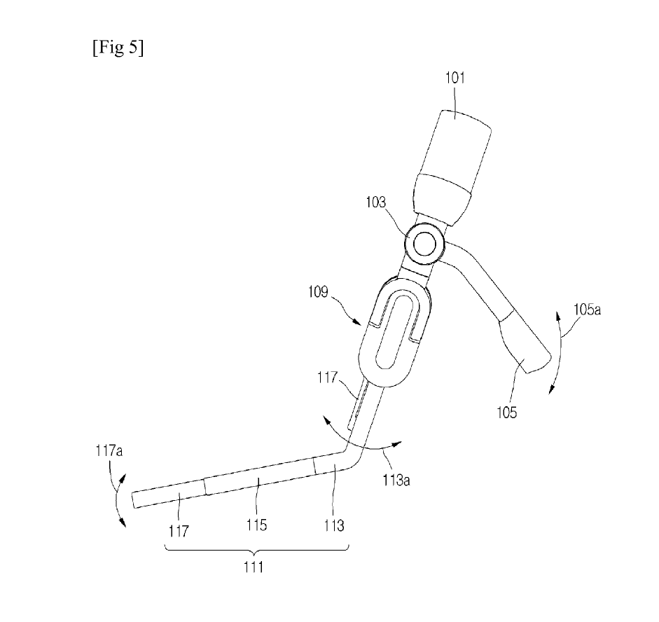

Referring to FIGS. 1 to 6, a detailed description of head gear for a tooth whitening apparatus according to an embodiment of the present disclosure will be given.

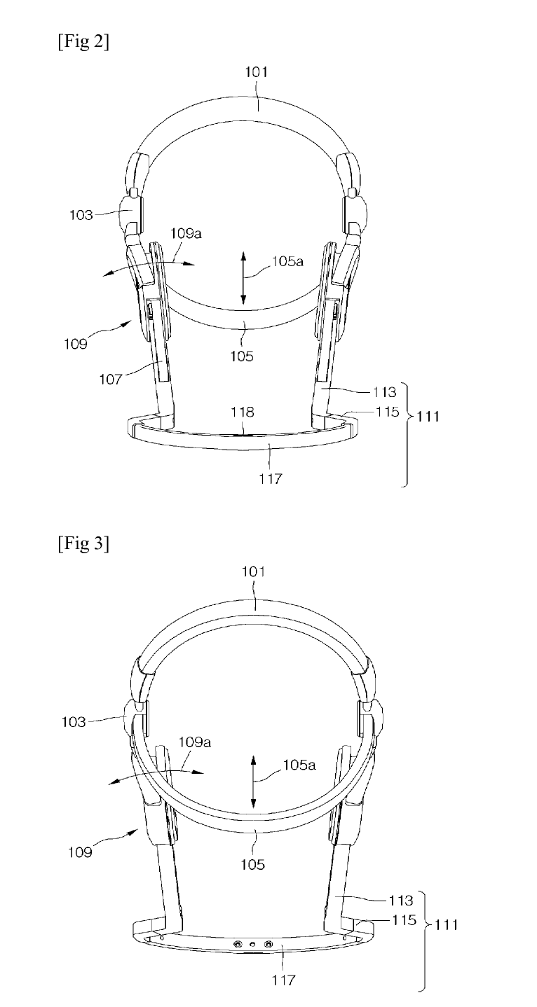

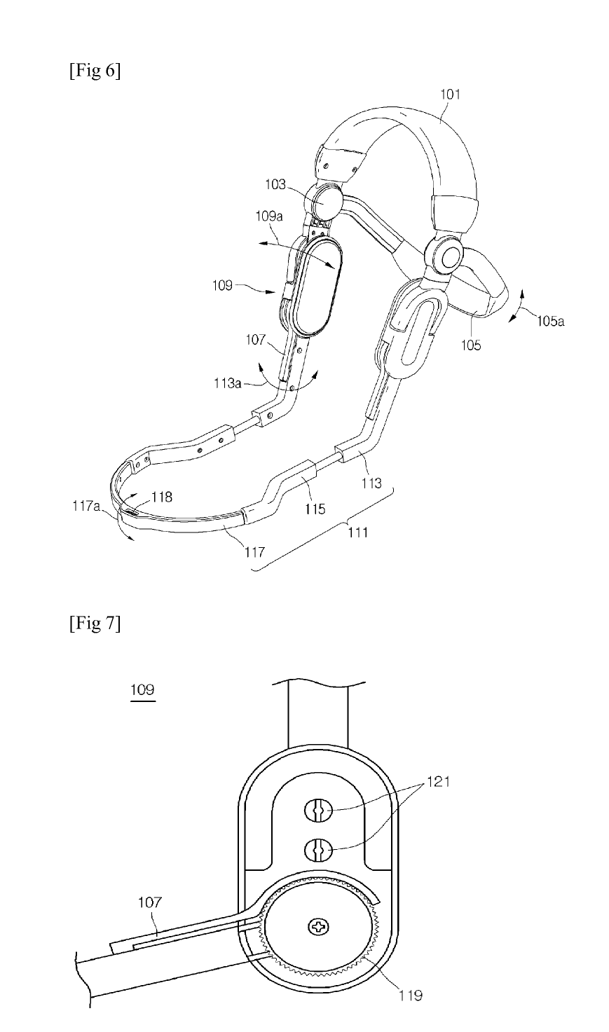

FIG. 1 illustrates a perspective view of head gear for a tooth whitening apparatus according to an embodiment of the present disclosure, and FIGS. 2 through 5 respectively illustrate a frontal view, a rear view, a top view, and a side view of the head gear in FIG. 1 as seen from directions A, B, C, and D. FIG. 6 illustrates a perspective view of the head gear in FIG. 1 illustrating a sliding portion extended outward.

Referring to FIGS. 1 to 5, head gear for a tooth whitening apparatus according to an embodiment of the present disclosure includes a top support band 101 that is fixed against the top of a patient's head, a rear support band 105 fixed against the posterior of the patient's head, a coupler 103 connecting the top support band 101 to the back support band 105, a side support 109 pressed against the temples, and a whitening apparatus support 111 connected to the side support 109 to hold a whitening apparatus installed thereon.

The top support band 101 is pressed against the top of the patient's head to fix the head gear on the patient's head. When the patient wears the head gear, the patient may move his/her neck and head without added securing devices and freely use both arms, and is free to roam within the range of a power cord. Pads for absorbing shock may be formed partially or entirely on the top support band 101, increasing the wearer's comfort level, and may employ flexible material such as elastic bands instead of stiff material.

The rear support band 105 is fixed against the rear of the patient's head, and fixes the head gear on the patient's head (like the top support band 101). Pads may be formed partially or entirely on the rear support band 105 to improve wearing comfort, and like the top support band, the rear support band 105 may be formed as a flexible rubber band instead of a hard material. The rear support band 105 may be connected to the top support band 101 through the coupler 103.

The rear support band 105 is pivotable about the axis of the coupler 103 in directions indicated by the double-sided arrow 105a (that is, in a vertical direction), and may be adjusted according to the size and shape of the patient's head.

The rear support band 105 does not have to be connected to the top support band 101 by means of the coupler 103. The rear support band 105 and the top support band 101 may be connected via other means, or do not have to be coupled to fix the top and rear of the patient's head.

The side support 109 presses against the patient's temples. It may be moved within a predetermined range in the direction of the arrows 109a to accommodate the size and shape of the patient's head. Pads with elasticity may be formed on contacting portion 110 of the side support 109 to improve wearing comfort.

According to embodiments, the top support band 101 and the rear support band 105 may be connected directly to the whitening apparatus support without the intervening side support 109.

Alternately, the side support 109 and the whitening apparatus support 111 may be provided without the top support band 101 or the rear support band 105. In this case, the whitening apparatus may be fixed on the patient's face by means of ear piece-type supports provided on the side support 109.

According to embodiments, speakers or bone conduction vibrating devices may be installed on the side supports 109 and connected to an MP3 audio device to enable a patient to enjoy music or radio programs while receiving tooth whitening treatment without any additional devices. As widely known, a bone conduction vibrating device does not directly transmit sound into an ear like an earphone, but transmits vibration to the temples to transmit sound, and is therefore a way of avoiding damage to the eardrums or hearing defects inflicted by the use of earphones. By installing a bone conduction vibrating device on the side support 109, a patient can experience less boredom during tooth whitening treatment while preventing damage to the hearing faculties.

Also, by installing a speaker or bone conduction vibrating device on the side support 109 and installing a display device in the hospital waiting room or treatment room, the patient is able to watch movies or TV.

Further, audio messages, such as “tooth whitening treatment is now beginning” or “there are 3 minutes remaining in your tooth whitening treatment”, may be broadcast to a patient via the speaker or bone conduction vibrating device on the side support 109.

In this way, not only are patients less bored, but announcements on the progress of tooth whitening treatments can be made to patients proximate to a broadcasting device.

According to some embodiments, a button for summoning a doctor or nurse may be provided on the side support 109 or another part of the head gear, or a microphone may be provided near a tooth whitening apparatus holder 117 or a slider 115 in order to allow the patient to deliver an audio message to a doctor or nurse. Thus, a high level of convenience can be afforded to both the patient and doctor during the long time it takes for a tooth whitening procedure.

FIG. 7 illustrates the inner structure of a side support 109. A rotating portion 119 may be formed in the shape of a serrated wheel, and a rotating button 107 may be disposed such that when it is not pressed, the serrated teeth and the rotating button 107 are engaged to fix the slider connector 113, and the slider connector 113 moves in a rotating direction 113a when the rotating button 107 is pressed to disengage from the teeth of the rotating portion.

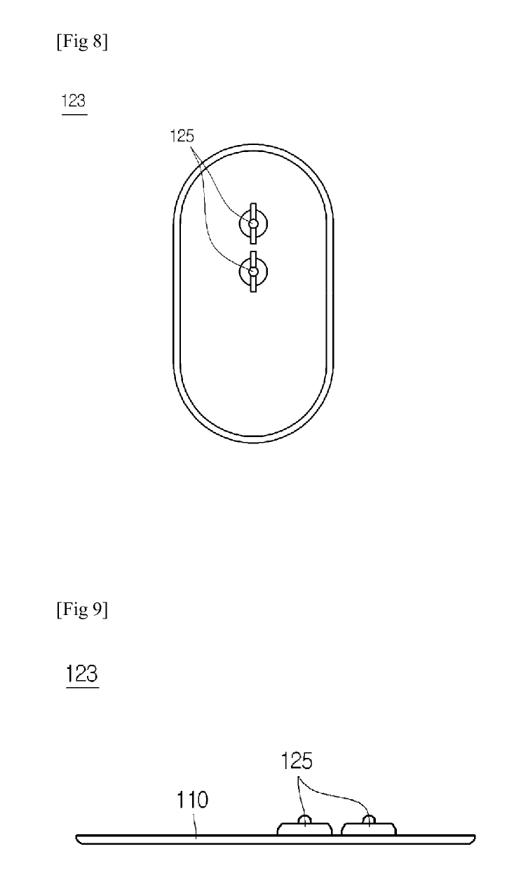

FIG. 8 is plan view of a cap 123 of a side support 109. The cap 123 for the side support is inserted and fixed to a fixing portion 121 of the side support 109 in FIG. 7. FIG. 9 illustrates a side view of the cap 123 in FIG. 8. Referring to FIG. 9, a contacting portion 110 that contacts the sides of a patient's head may be provided on the cap 123, and the contacting portion may have a pad that improves wearing comfort.

As described above, the side support 109 may include a speaker, bone conduction vibrating device, earphone, or other device together with the contacting portion.

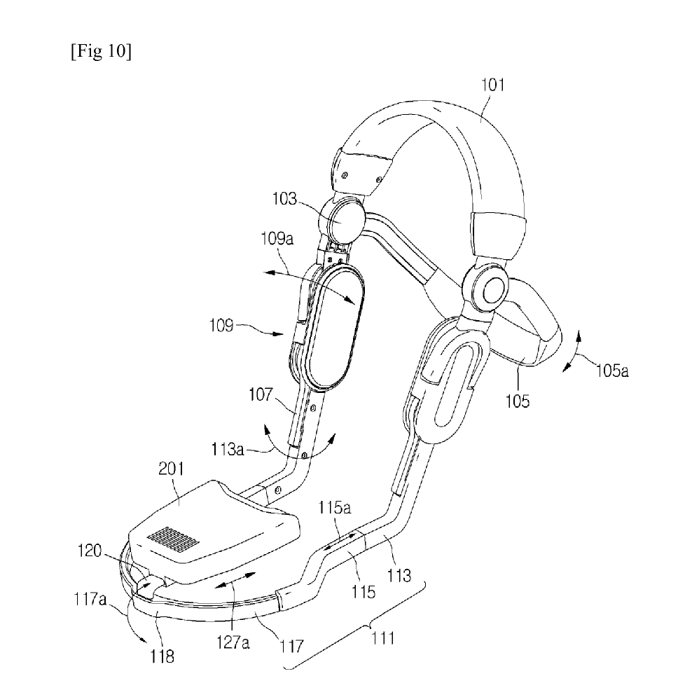

The whitening apparatus support 111 is a portion on which a tooth whitening apparatus 201 is installed, as illustraten in FIG. 10, and includes the slider connector 113 connected to the side support 109, the slider 115 connected to the slider connector 113, and the tooth whitening apparatus holder 117 connected to the slider and having the tooth whitening apparatus installed thereon.

The slider connector 115 is connected to the side support 109, and is rotatable in the rotating direction indicated by the arrow 113a by pressing the rotating button 107. By rotating the slider connector, the position of the tooth whitening apparatus installed on the whitening apparatus holder 117 can be adjusted to contact a patient's mouth. The slider 115 may be connected to the slider connector 113 to be slidably extendable in the direction indicated by the arrow 115a. FIG. 6 illustrates the slider 115 in an extended state. By extending the slider 115, the head gear may be adjusted according to the size and shape of a patient's head and jaw.

A slot 118 is provided in the whitening apparatus support 117 to enable the tooth whitening apparatus 201 to be installed, as illustraten in FIG. 10. Referring to FIG. 10, a whitening apparatus connector 120 in the form of rail is provided at the bottom of the tooth whitening apparatus 201, and the whitening apparatus connector 120 is removably inserted into the slot 118 on the whitening apparatus support 117. Thus, the tooth whitening apparatus may be precisely put into contact with a patient's mouth in accordance with the size and shape of the patient's head by sliding the whitening apparatus support 117 with the tooth whitening apparatus installed thereon forward or backward with respect to the patient—that is, in the direction of the arrow 127a.

The whitening apparatus support 117 is capable of being disengaged from the slider 115, enabling a patient to apply tooth whitening material on the patient's teeth without being impeded.

Likewise, the tooth whitening apparatus 201 is also capable of being removed from the whitening apparatus support 117, so that it may be removed while applying whitening material and reinstalled to be used for tooth whitening treatment.

As illustraten in FIG. 10, the slot 118 in which the tooth whitening apparatus is installed may be rotated in the direction of the arrow 117a independently of other portions, so that it will not interfere when applying whitening material on teeth.

During tooth whitening treatment, a patient applies the tooth whitening apparatus 201 against his/her mouth with the mouth slightly open. According to embodiments, in order to completely seal the tooth whitening apparatus 201 with a patient's mouth, a mouthpiece may be inserted in the patient's mouth and the tooth whitening apparatus 201 may be fixed to the mouthpiece.

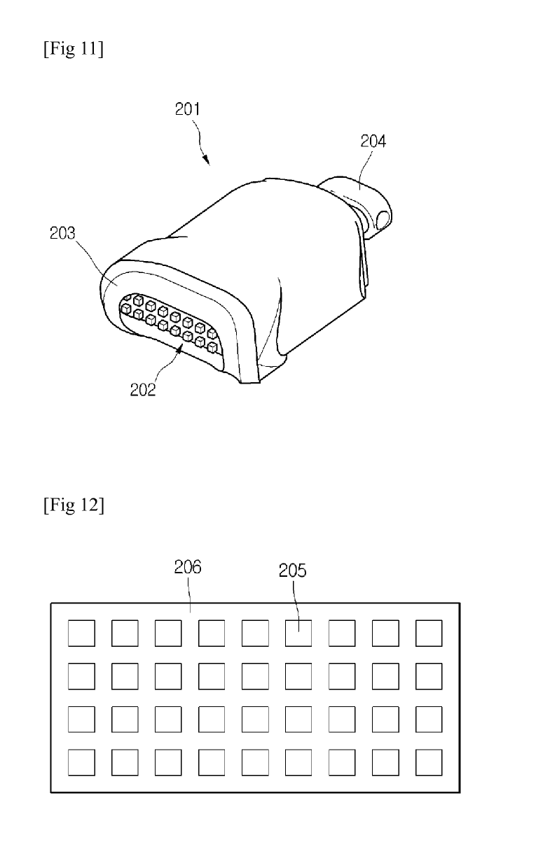

FIG. 11 illustrates a perspective view of the tooth whitening apparatus 201 according to the present disclosure. As illustraten in FIG. 10, the tooth whitening apparatus 201 may be detachably installed on the whitening apparatus support 117 through the whitening apparatus connector 120.

A tooth whitening apparatus 201 according to the present disclosure includes a light emitting portion 202 that has a plurality of chip light emitting diodes (LED) arranged to emit light on the teeth, a mouth sealing portion 203 for sealing a patient's mouth against the tooth whitening apparatus, and a coupling portion 204 for coupling the tooth whitening apparatus 201 to the supporter.

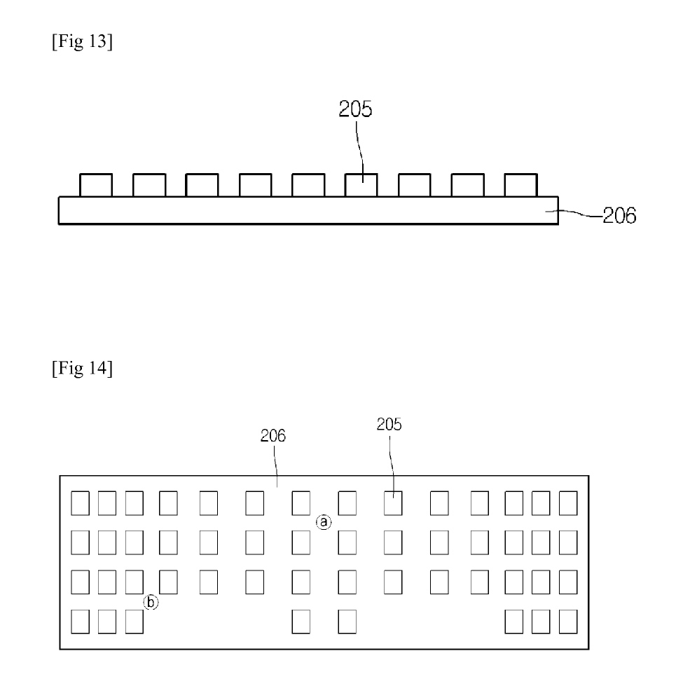

FIGS. 12 and 13 respectively illustrate a plan view and a sectional view of a light emitting portion 202.

As illustrated in FIG. 12, the light emitting portion 202 is formed of a plurality of light emitting devices 205 arranged on a printed circuit board (PCB) 206.

The light emitting devices 205 may be chip LEDs.

LEDs include many types such as ultra bright diffuser LEDs and hyper LEDs used for indoor lighting, automotive brake lights, etc. Of these, chip LEDs are capable of being mounted on a substrate like an integrated circuit (IC) chip, are smaller than other LED types, and are light emitting devices that are used as backlights for liquid crystal displays (LCD) in cellular phones, MP3 players, etc.

The size of chip LED used as the light emitting device 205 according to the present disclosure may differ according to different embodiments, and may be the same size or smaller than a person's tooth. Preferably, the size of a chip LED used as the light emitting device 205 may be 3.5 mm×2.8 mm and have a thickness of 0.4 mm.

The angular range of light emitted from the light emitting device 205 according to the present disclosure may be approximately 120°-130° in the direction in which light from the light emitting device 205 is emitted.

The light emitted from the chip LED according to the present disclosure is visible light with a wavelength range between 380 nm-520 nm, and may be a blue light with a 460 nm wavelength.

The reason for using visible light is that when tooth whitening material is applied to teeth and light is emitted during tooth whitening treatment, the visible rays are more effective at quickly dissolving the whitening material so that the ingredients for tooth whitening can more effectively penetrate the tooth surfaces.

According to different embodiments, the light emitting device 205 used according to the present disclosure may be a metal halide lamp instead of a chip LED.

According to different embodiments, a heat dissipating fan may be installed to dissipate heat generated from the light emitting device 205.

The chip LED used as the light emitting device 205 of the tooth whitening apparatus 201 according to the present disclosure uses a low current of 50 mA-150 mA, that is (depending on the type of whitening material used) capable of satisfactory tooth whitening when emitted for a duration of approximately 5 minutes or more at a distance of 1-4 cm from the patient's teeth.

Also, depending on the condition of the patient's teeth, the current supplied to the light emitting device 205 of the tooth whitening apparatus 201 may be adjusted to reduce the tooth whitening treatment time, or emit a suitable intensity of light to obtain suitable results for the condition of the particular patient's teeth.

According to embodiments, a timer may be installed to control the current supplied to the light emitting device 205, in order to automatically turn off power when a preset whitening duration elapses.

If the current supplied to the light emitting device 205 of the tooth whitening apparatus 201 according to the present disclosure is increased to approximately 300 mA-350 mA, the apparatus may be used as a curing apparatus that photopolymerizes dental resin used for filling cavities and other dental resins for teeth. When dental resin is applied to a patient's tooth, and light is emitted for approximately 1 minute at a distance of less than 1 cm from the resin at an intensity of approximately 300 mA-350 mA, sufficient hardening can be obtained.

A factor to consider is that a patient's front teeth are closer to the tooth whitening apparatus 201 than the molars, which are further away. Thus, a weaker current may be supplied to the chip LEDs disposed at the center of a substrate 206 corresponding to the front teeth, and a stronger current can be supplied to the chip LEDs corresponding to the molars at the edges of the substrate 206, in order to obtain even whitening results overall.

FIG. 14 illustrates a light emitting portion 202 of a tooth whitening apparatus according to other embodiments of the present disclosure.

The light emitting devices are more densely arranged in the edge region D where the molars are located than in the central region D where the front teeth are located, and the edge region D is located farther away from the teeth than the central region D. When light is emitted uniformly, light is concentrated in the central region D. Thus, taking the latter into account, the density of the light emitting devices 205 at the central region D is made less than that in the edge region D, in order to improve uniformity in tooth whitening.

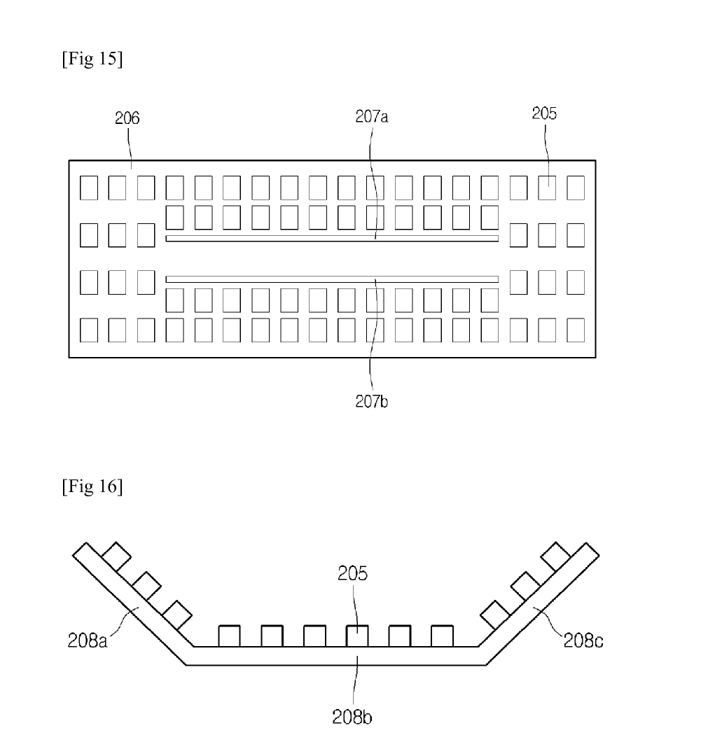

FIG. 15 illustrates a plan view of a light emitting portion 202 according to another embodiment.

A patient receives tooth whitening treatment with his/her mouth slightly open. However, when the mouth is open, there is an empty space in the center between the top and bottom teeth, where there is no need to emit light. Taking this into account, the mirrors 207a and 207b are disposed at the center of the tooth whitening portion to reflect light emitted toward the center of the mouth upwards and downwards and augment the effects of tooth whitening.

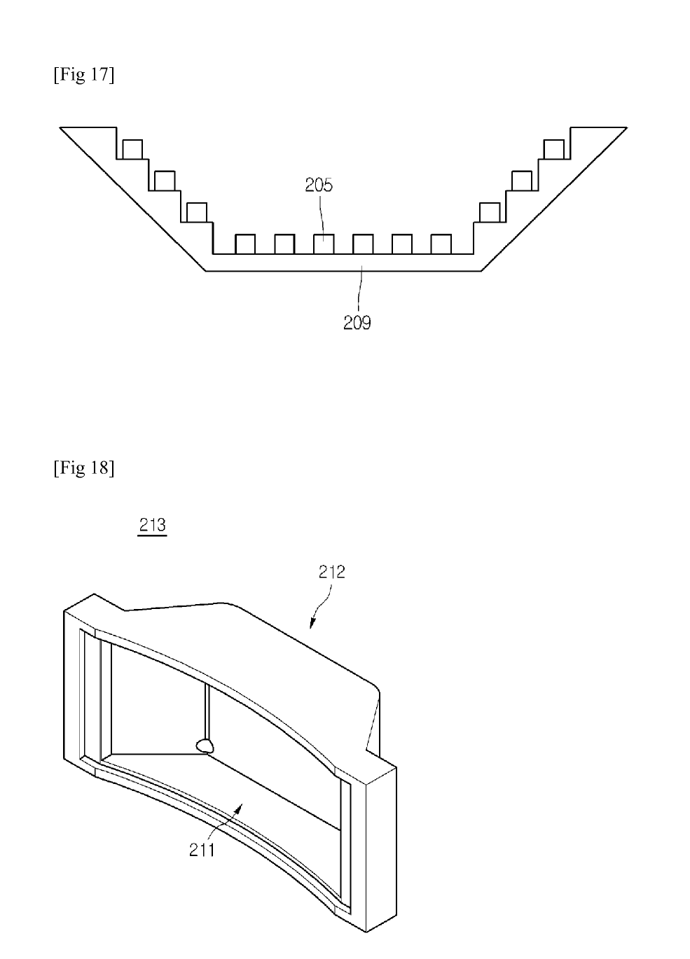

FIGS. 16 and 17 respectively illustrate a sectional view of a light emitting portion 202 of a tooth whitening apparatus according to other embodiments of the present disclosure.

Referring to FIG. 16, when considering the shape of a human mouth, light emitting devices 205 may be arranged on a PCB consisting of three plates 208a, 208b, and 208c, instead of on a flat PCB.

The light emitting devices on the side PCBs 208a and 208c largely emit light onto the molars, and those on the middle PCB 208b largely emits light onto the front teeth and canines By thus arranging the light emitting devices, the central region and the edge regions of human teeth can receive light more uniformly.

According to embodiments, a stronger current may be supplied to light emitting devices on the edge regions 208a and 208c of the PCB, and a weaker current may be supplied to light emitting devices on the central region 208b of the PCB to realize uniform whitening of teeth.

Also, as illustraten in FIG. 17, the PCB 209 may have a stepped formation. In the structure illustraten in FIG. 16, light emitted from the light emitting devices 205 is incident perpendicularly on teeth to increase effectiveness of tooth whitening.

According to embodiments, the light emitting devices 205 used by the tooth whitening apparatus 201 may be metal halide lamps instead of chip LEDs.

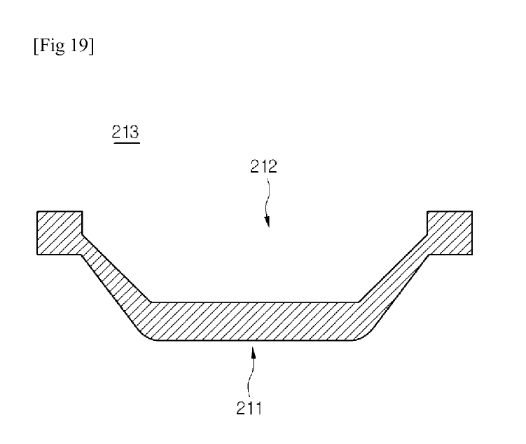

FIGS. 18 and 19 respectively illustrate a perspective view and a sectional view of a body 213 of a light emitting portion 202 forming a tooth whitening apparatus 201 according to other embodiments of the present disclosure. The body 213 is disposed within the case of the tooth whitening apparatus 201 illustraten in FIG. 11, and functions as a frame for the tooth whitening apparatus 201 with the PCB illustraten in FIGS. 12 to 15 and 16 and 17 installed therein.

The body 213 may be made of a metal—particularly, aluminum. A PCB on which light emitting devices are mounted—that is, the light emitting portion 202—is mounted to the front 211 of the body 213, and at the rear, a heat sink (not illustraten) that dissipates heat generated by the light emitting devices 205 or a heat dissipating fan (not illustraten) may be disposed to the rear 212 of the body 213.

A power supply (not illustraten) that supplies direct current to the light emitting portion of the tooth whitening apparatus 201 may be required, in which case power may be drawn from an external source through a cable. Alternately a disposable or rechargeable battery may be installed within the tooth whitening apparatus 201 as a power source. However, because installing a battery inside the tooth whitening apparatus 201 would make the apparatus heavy and therefore uncomfortable for a patient to wear during tooth whitening treatment, the use of an external power supply through a cable may be preferable.

The supplying of power to and the controlling of the tooth whitening apparatus 201 during tooth whitening treatment is performed by a controller (not illustraten) connected to the tooth whitening apparatus 201.

One controller may be connected to a plurality of tooth whitening apparatuses 201, enabling a dentist to perform tooth whitening treatment simultaneously on a number of patients. Of course, the one controller should be capable of controlling the tooth whitening duration, intensity, etc. of the respective tooth whitening apparatuses.

While the present disclosure has been described and illustrated herein with reference to the preferred embodiments thereof, and specific language has been used to facilitate a general technical understanding thereof, the present disclosure is not limited thereto. Here, it will be apparent to those skilled in the art that various modifications and variations can be made therein without departing from the spirit and scope of the disclosure. Thus, it is intended that the present disclosure covers the modifications and variations of this disclosure that come within the scope of the appended claims and their equivalents.

1. A head gear for a tooth whitening apparatus, the head gear comprising:
a side support contacting a side of a head;
a tooth whitening apparatus support connected to the side support and on which the tooth whitening apparatus is installed;
a top support contacting and fixed on a top of the head;
a rear support contacting and fixed at a rear of the head; and
a coupler connecting the top support to the rear support.

2. (canceled)

3. (canceled)

4. The head gear according to claim 1, wherein the rear support is capable of rotating upward and downward about the coupler.

5. The head gear according to claim 1, wherein the tooth whitening apparatus support comprises:
a slider connector connected to the side support;
a slider connected to the slider connector; and
a tooth whitening apparatus holder connected to the slider and on which the tooth whitening apparatus is installed.

6. The head gear according to claim 5, wherein the slider is extendably connected to the slider connector.

7. The head gear according to claim 5, wherein the tooth whitening apparatus holder is detachably connected to the slider.

8. The head gear according to claim 5, wherein the tooth whitening apparatus holder is capable of rotating with respect to the slider.

9. The head gear according to claim 1, wherein the side support adjustably contacts the side of the head.

10. The head gear according to claim 1, wherein the side support comprises a speaker or a bone conduction vibrating device installed thereon.

11. (canceled)

12. (canceled)

13. (canceled)

14. (canceled)

15. (canceled)

16. (canceled)

17. (canceled)

18. (canceled)

19. A tooth whitening set comprising:
a side support contacting a side of a head;
a tooth whitening apparatus support connected to the side support; and
a tooth whitening apparatus installed on the tooth whitening apparatus support to radiate light on teeth.

20. The tooth whitening set according to claim 19, wherein the tooth whitening apparatus is capable of moving forward and backward when installed on the tooth whitening apparatus support.

21. The tooth whitening set according to claim 19, further comprising: a top support contacting and fixed on a top of the head; and a rear support contacting and fixed at a rear of the head.

22. The tooth whitening set according to claim 21, further comprising a coupler connecting the top support and rear support.

23. The tooth whitening set according to claim 19, wherein the tooth whitening apparatus comprises:
a flat panel type substrate;
a plurality of chip LEDs mounted on the substrate; and
a power supply supplying power to the chip LEDs.

24. The tooth whitening set according to claim 19, wherein the tooth whitening apparatus comprises:
a substrate formed of a plurality of surfaces capable of enclosing a human mouth;
a plurality of chip LEDs mounted on each of the surfaces; and
a power source supplying power to the chip LEDs.

25. The tooth whitening set according to claim 23, wherein a current supplied to chip LEDs mounted on sides of the substrate from the plurality of chip LEDs is more intense than a current supplied to chip LEDs mounted on a middle of the substrate from the plurality of chip LEDs.

26. The tooth whitening set according to claim 24, wherein a current supplied to chip LEDs mounted on sides of the substrate from the plurality of chip LEDs is more intense than a current supplied to chip LEDs mounted on a middle of the substrate from the plurality of chip LEDs.
